(19) United States
(12) Patent Application Publication
(10) Pub. No.: US 2014/0120551 A1
(43) Pub. Date: May 1, 2014

(54) METHODS OF DIAGNOSING, PREDICTING AND TREATING CARDIOVASCULAR DISEASE

(71) Applicant: ACADEMIA SINICA, TAIPEI CITY (TW)

(72) Inventors: RUEY-BING YANG, TAIPEI (TW); TE-FA CHIU, TAOYUAN (TW)

(73) Assignee: ACADEMIA SINICA, TAIPEI CITY (TW)

(21) Appl. No.: 14/064,511

(22) Filed: Oct. 28, 2013

Related U.S. Application Data
(60) Provisional application No. 61/719,654, filed on Oct. 29, 2012.

(51) Int. Cl. G01N 33/68

(52) U.S. Cl. CPC G01N 33/6893
USPC 435/7.4; 436/501; 435/7.92

(57) ABSTRACT

Methods for determining an increased risk of developing a cardiovascular event excluding stable angina in a subject are disclosed. Also disclosed are methods for diagnosing a human subject's myocardial infarction (MI) and/or acute coronary syndrome (ACS) state or identifying a human subject's risk of MI and/or ACS.

### REFERENCES TO RELATED APPLICATION

The present application claims priority to U.S. Provisional Application Ser. No. 61/719,654 filed Oct. 29, 2012, which is herein incorporated by reference in its entirety.

### FIELD OF THE INVENTION

The present invention relates generally to cardiovascular disease.

### BACKGROUND OF THE INVENTION

Heat-shock proteins (HSPs) are abundant intracellular proteins found in both prokaryotic and eukaryotic organisms. Most HSPs act as chaperones and are involved in protein folding and transport. HSPs could be grossly classified by molecular mass into the general HSP family (˜40-110 kDa) or the small HSP family (sHSP, ˜10-30 kDa). The general HSPs, including HSP60, 70 or 90, are associated with cardiovascular diseases including cardiac hypertrophy, heart failure and ischemia/reperfusion injury. Therefore, general HSPs may contribute to the protective mechanism during the pathogenesis of cardiac hypertrophy, heart failure, and ischemia/reperfusion injury. However, the involvement of sHSPs in cardiovascular diseases remains largely unknown.

HSPB7, a sHSP, was first identified by Krief et al (1999) and designated as a cardiovascular HSP for its high expression in the heart, low expression in the skeletal muscle but virtual absence in all other tissues examined. HSPB7 is characterized by its small molecular mass, ˜20 kDa, and a highly-conserved α-crystalline domain. Sub-cellular fractionation and confocal immunofluorescence analysis showed that sHSPs including HSPB7 are localized within the cytosol or associated with myofibrils in cardiac or skeletal muscle cells. Despite its abundant cardiac expression, HSPB7 was recently linked to sporadic heart failure by genome-wide association studies (Cappola T P et al. 2010; Matkovich S J et al. 2010; Stark K et al. 2010). However, whether HSPB7 is involved in the pathogenesis of coronary artery disease (CAD) or acute coronary syndrome (ACS) remains uninvestigated.

### SUMMARY OF THE INVENTION

In one aspect, the invention relates to a method for determining an increased risk of developing a cardiovascular event excluding stable angina in a subject, comprising:

a) obtaining a plasma sample from the subject;

b) measuring a plasma level of heat shock protein B7 (HSPB7) in the sample;

c) comparing the plasma level of HSPB7 in the sample with a control; and

d) determining that the subject has an increased risk for developing the cardiovascular event excluding stable angina if the plasma level of HSPB7 in the sample is higher than the control.

In another aspect, the invention relates to a method for diagnosing a human subject's myocardial infarction (MI) and/or acute coronary syndrome (ACS) state or identifying a human subject's risk of MI and/or ACS, the method comprising:
a) measuring the level of heat shock protein B7 (HSPB7) in a plasma sample from said human subject:
b) comparing the level of heat shock protein B7 (HSPB7) in the plasma sample from said subject to data obtained from one or more than one reference plasma sample from a control human subject to identify an increase or decrease in the level of HSPB7 in the plasma sample from said subject; and
c) using said increase or decrease in the level of HSPB7 in the plasma sample from said human subject to diagnose the human subject's MI and/or ACS state or change in MI and/or ACS health state, or to identify the risk of MI and/or ACS in said subject, wherein the human subject is diagnosed with MI and/or ACS or an increased risk of MI and/or ACS based on having an elevated level of HSPB7.

The analyzing step may further comprises measuring the level of cardiac troponin 1 (cTn1) or creatinine kinase-MB isoform (CK-MB), or both, and wherein the human subject is diagnosed with MI and/or ACS based on having an elevated level of cTn1 or CK-MB, or both.

Further in another aspect, the invention relates to a method for diagnosing and/or predicting ACS in a human subject, comprising:

a) measuring a plasma level of HSPB7 in the human subject;

b) comparing the plasma level of HSPB7 in the human subject to the plasma level of HSPB7 of a control human subject; and

c) diagnosing or predicting relative risk for the development of ACS in said human subject based on having an elevated level of IHSPB7.

In one embodiment of the invention, the measuring step is performed under 24 hours from receiving a complaint of chest pain from the subject.

In another embodiment of the invention, the measuring step is performed within 20 hours from receiving a complaint of chest pain from the subject, and wherein the cardiovascular event is myocardial infarction.

In another embodiment of the invention, the measuring step is performed within 12 hours from receiving a complaint of chest pain from the subject.

In another embodiment of the invention, the measuring step is performed within 6 hours from receiving a complaint of chest pain from the subject.

In another embodiment of the invention, the measuring step is performed under 6-12 hours from receiving a complaint of chest pain from the subject.

In another embodiment of the invention, the measuring step is performed within 3 hours from receiving a complaint of chest pain from the subject.

In another embodiment of the invention, the measuring step is performed within 1-3 hours from receiving a complaint of chest pain from the subject.

In another embodiment of the invention, the determining step identifies t that the subject has an increased risk for developing acute coronary syndrome (ACS) if the plasma level of HSPB7 in the sample is ≧5.1 ng/mL.

In another embodiment of the invention, the method further comprises measuring a plasma level of cardiac troponin I (cTnI) or creatinine kinase-MB isoform (CK-MB), or both; comparing the plasma level of cTn1 or CK-MB with corresponding data from a control; and identifying the subject as having ACS if the plasma levels of cTn1, CK-M or both are higher than the control.

In another embodiment of the invention, the plasma level of HSPB7 in the sample is ≧2 ng/mL.

In another embodiment of the invention, the cardiovascular event is selected from the group consisting of atherosclerotic vascular disease, myocardial infarction, acute cardiac syndrome, stroke, a transient ischemic attack, and critical limb ischemia.

In another embodiment of the invention, the measuring step is performed with an enzyme-linked immunosorbent assay (ELISA) using anti-human HSPB7 monoclonal antibody (mAb).

Further in another embodiment of the invention, the anti-hHSP7 mAb recognizes an epitope located in amino acid residues 2-170 of a human HSPB7.

Yet in another embodiment of the invention, the subject is free of any one of the following conditions or diseases: idiopathic cardiomyopathic conditions, significant valvular heart disease, any malignancy, hematologic or rheumatologic disease, and chronic kidney disease.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 shows production and specificity of anti-HSPB7 specific monoclonal antibodies (mAbs). Four independent hybridoma clones (mAbs 2, 3, 5, and 6) were generated in BALB/c mice immunized with the recombinant HSPB7 protein. Each of these mAbs could specifically recognize the epitope HA-tagged HSPB7 (HA.HSPB7) expressed in HEK-293T cells but not HSPB9 (the most closely related member of sHSP family) (panel A). As a control for protein loading, protein expression of the HA-tagged HSP proteins was confirmed by anti-HA antibody (bottom panel). In addition, these Anti-HSPB7 mAbs can recognize HSPB7 from the human (h) or the mouse (m) but not from the zebrafish (z) (panel B). As a control for protein expression, the HA-tagged human HSPB7, FLAG-tagged mouse or zebrafish HSPB7 was confirmed by the corresponding anti-HA or anti-FLAG antibody, respectively. C, HSPB7 expression in the heart by immunohistochemistry. Cross-section of the mouse heart was stained with the anti-HSPB7-specific mAb 2. HSPB7 immunoreactivity was predominantly detected in the cardiomyocytes. Scale bar: 50 μm. WB, western blot analysis; IHC, immunohistochemical analysis.

FIG. 2 shows plasma concentration and protein expression of HSPB7 after coronary artery ligation in mice. (A) Plasma concentration of HSPB7 was determined after myocardial infarction (MI) at 1, 3, 6, 12, 24 and 48 h induced by coronary artery ligation in mice. Data are means±SEM (n=8-11 animals in MI group; and n=6 in the sham group). *, p<0.05 versus the sham group. (B) Western blot analysis of myocardial HSPB7 expression after MI treatment or sham control. (C) Immunohistochemical staining analysis of HSPB7 expression on the heart sections from sham control or MI animals. Inserts indicates the images at low magnifying power. Arrowhead indicates the infarct.

FIG. 3 shows plasma concentration of HSPB7 in control patients (non-cardiac chest pain) and those with stable angina and acute coronary syndrome chest pain. Plasma concentration of HSPB7 is significantly higher in patients with acute coronary syndrome than patients with stable angina (p<0.001) and control patients (non-cardiac chest pain) (p<0.001).

FIGS. 4A-B show correlation of plasma HSPB7 concentration and levels of cardiac troponin 1 (cTn1; FIG. 4A) or creatinine kinase-MB isoform (CK-MB, FIG. 4B) concentration. Data are logarithmically transformed levels of HSPB7, cTn1, and CK-MB.

FIG. 5 is a scatterplot showing the relation between initial plasma HSPB7 concentration and time after symptom onset. One point represents 1 subject. Plasma HSPB7 level was detected as early as 3 h after symptom onset but no later than 24 h in patients with ST-elevation myocardial infarction.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The terms “HSPB7” and “HspB7” are interchangeable, and stand for “heat shock protein beta-7”. The alternative name is “cardiovascular heat shock protein”. The amino acid sequences of heat shock protein beta-7 (HspB7) of human, mouse and zebra fish species are SEQ ID NOs: 1-3, respectively.

As used herein, the term “control” generally means a reference obtained from a subject without a cardiovascular event.

As used herein, the term “cardiovascular event” generally refers to one of the following conditions: atherosclerotic vascular disease, myocardial infarction, acute cardiac syndrome (ACS), stroke, a transient ischemic attack, and critical limb ischemia, but excludes the following conditions or diseases: idiopathic cardiomyopathic conditions, significant valvular heart disease, any malignancy, hematologic or rheumatologic disease, and chronic kidney disease.

The invention is related to the discovery that the expression of HSPB7 is affected and HSPB7 is released into the circulation during myocardial ischemia or after myocardial necrosis. We have investigated the potential association of plasma HSPB7 concentration with ACS in both an animal model and human subjects. It was found that the plasma concentration of HSPB7 was rapidly elevated after myocardial infarction and was an independent predictor of ACS.

### EXAMPLES

### Methods

### Animal and Human Protocols

All surgical procedures were performed according to the protocols approved by the Institutional Animal Care and Utilization Committee, Academia Sinica. The investigation complied with the Guide for the Care and Use of Laboratory Animals published by the US National Institute of Health (NIH Publication No. 85-23, revised 1996). Investigations involving human subjects were conducted in compliance with the principles outlined in the Declaration of Helsinki.

Generation of Anti-HSPB7 Monoclonal Antibodies (mAbs)

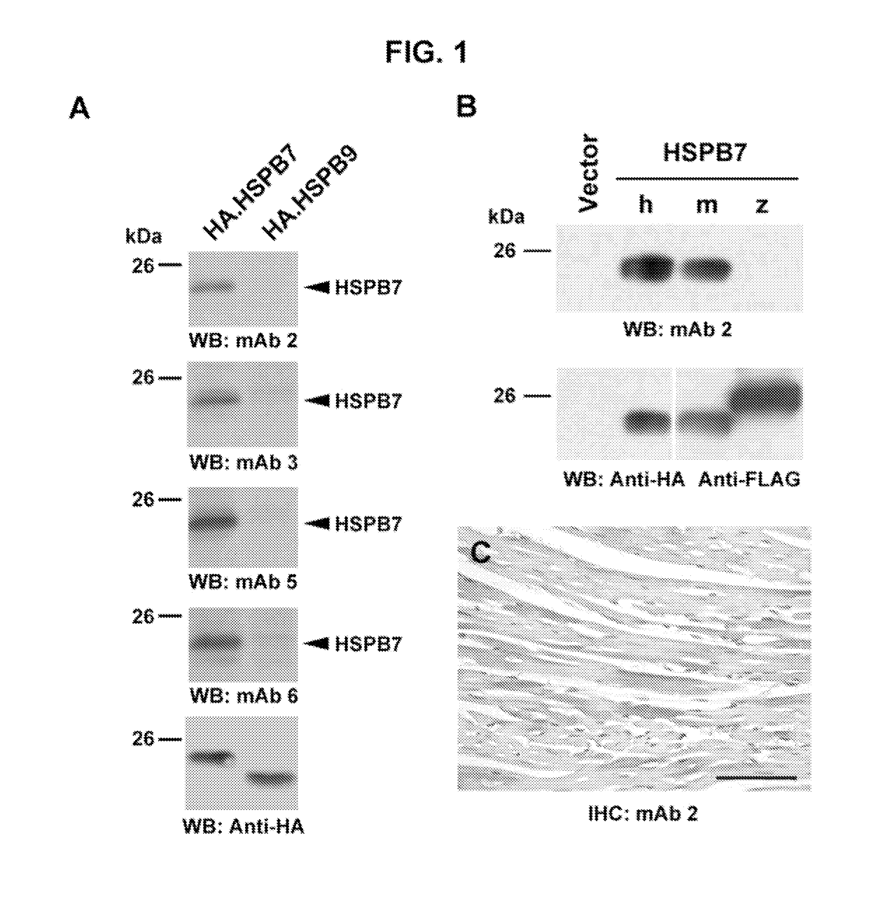

Splenocytes from BALB/c mice immunized with recombinant GST-HSPB7 (residues 2-170) protein were fused with P3X myeloma cells to produce hybridomas. Hybridomas positive for HSPB7 were identified and cloned. Ascites fluids were prepared, and purified IgG was obtained by protein G chromatography. We obtained 4 independent clones, mAb 2, 3, 5, and 6, that could specifically recognize the recombinant full-length HSPB7 protein expressed in human embryonic kidney (HEK)-293T cells by western blot analysis (FIG. 1). The mAbs 2 and 5 were used in following experiments because of their higher specificity.

### Animal Model of Myocardial Infarction (MI)

Coronary artery ligation was used as an animal model of myocardial infarction as described (Tarnavski O et al. 2004). The incision landmark was the left armpit. An oblique 8-mm incision was made 2 mm away from the left sternal border toward the left armpit. The muscles were separated without damaging blood vessels. After the opening of the chest cavity, the chest retractor was applied for better visualization of the pericardium. The left anterior descending (LAD) coronary artery could be visualized after opening the pericardium. The LAD artery was ligated 1 to 2 mm with a 7-0 silk ligature below the tip of the left auricle in its normal position. Occlusion was confirmed by the change of color of the myocardium. The chest wall was closed layer by layer. Mice in the sham control group underwent the entire procedure except for ligation of coronary artery. Blood samples were collected by laparotomy via the inferior vena cava for HSPB7 assay at indicated time points after MI.

### Patient Enrollment

The collection of patient samples was approved by Institute of Review Board of Chang-Gung Memorial Hospital, Taoyuan, Taiwan (No. 97-0606C) and the Institute of Biomedical Sciences, Academia Sinica, Taipei, Taiwan (No. AS-IRB01-10050 (07010)). Adult patients (age ≧18 years old) presenting to the emergency department with chest pain as the chief complaint were evaluated for eligibility. Exclusion criteria included idiopathic cardiomyopathic conditions, significant valvular heart disease, any malignancy, hematologic or rheumatologic disease, and chronic kidney disease (serum creatinine kinase level ≧3 mg/dL). Patients gave their informed consent to be in the study. Blood samples were collected for laboratory testing for blood count, lipid profile and levels of creatinine kinase-MB isoform (CK-MB), cardiac troponin I (cTnI), and HSPB7. Patients were assigned to the non-cardiac chest pain group, stable angina (SA) group, or ACS group by American College of Cardiology (ACC)/American Heart Association (AHA) guidelines for the management of ST-elevated myocardial infarction and unstable angina/non-ST-elevated myocardial infarction (Anderson J L et al. 2007).

### Plasma HSPB7 Assay

Heparinized blood samples were centrifuged at 2,500 g, and plasma was stored at −80° C. for further analysis. An in-house capture enzyme-linked immunosorbent assay (ELISA) by using anti-HSPB7 mAb 2 and 5 was developed as described (Dai D F et al. 2008). The minimum detection limit by this method was 2 ng/mL. All assays were performed in batch by another investigator blinded to the clinical diagnoses. The intra-assay and inter-assay covariance of this assay was less than 10%.

### Statistical Analysis

Demographic data are presented as mean±SD for continuous variables and number (percentages) for binominal variables. Analysis of the baseline characteristics of participants followed a case-control design, namely, ACS versus control subjects and SA versus control subjects. The chi-square and two-sample t test/Mann-Whitney rank sum tests were used for analysis. Circulating HSPB7 levels exhibited a log normal distribution, and therefore data were transformed (log 10) before logistic regression analysis. Multiple logistic regression analysis was used to evaluate the association(s) between HSPB7 concentration and ACS or risk factors, with appropriate adjustments for covariates. A two-tailed P<0.05 was considered statistically significant. Analyses involved use of SAS v9.1 (SAS Inst., Cary, N.C., USA).

### Results

Production of Anti-HSPB7-Specific Monoclonal Antibodies (mAbs)

To investigate the clinical association of cardiac HSPB7 at the protein level, we first generated mAbs against human HSPB7 protein. Spleen cells from BALB/c mice immunized with the GST-HSPB7 fusion protein containing residues 2-170 of human HSPB7 were used to prepare the mAb with use of a standard hybridoma technique (see Methods). After screening by ELISA and subcloning, 4 specific mAbs clones (2, 3, 5, and 6) against human HSPB7 were obtained. The specificity of these mAbs was tested by western blot analysis with recombinant human HSPB7 or HSPB9 protein (the most closely related sHSP family member) expressed in HEK-293T cells. As shown in FIG. 1A, these mAbs detected only human HSPB7 and did not cross-react with human HSPB9. In addition, these mAbs also recognized mouse HSPB7 protein by western blot or validated its expression in the cardiomyocytes by immunohistochemical analysis (FIGS. 1B and C). Te-Fa Chiu et al “Association of Plasma Concentration of Small Heat Shock Protein B7 With Acute Coronary Syndrome” Circ J 2012; 76: 2226-2233, which is incorporated herein by reference in its entireties.

### Profile of Released HSPB7 in Mouse Model of Myocardial Infarction (MI)

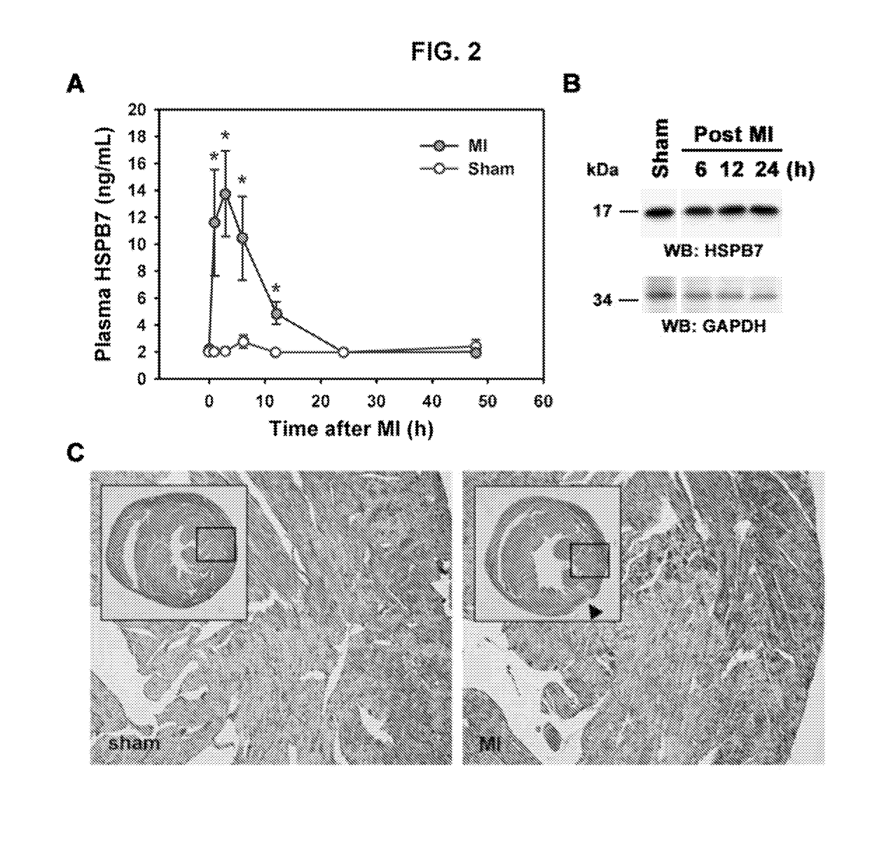

We then examined whether myocardial HSPB7 can be released into the circulation in a mouse model of MI induced by ligation of the LAD coronary artery (Tarnavski O et al. 2004). In MI mice, the plasma HSPB7 level was significantly elevated as early as 1 h after LAD ligation, peaked at 6 h and remained detectable up to 12 h after MI (FIG. 2). Plasma HSPB7 concentration returned to the minimal detection threshold level at 24 or 48 h after MI. However, in the sham control animals, HSPB7 concentration was below or around the minimal detection level at all time points (FIG. 2A). In addition, the expression of myocardial HSPB7 protein remains basically unaltered before or after MI as determined by Western blotting (FIG. 2B) or immunohistochemical staining for HSPB7 (FIG. 2C), respectively. Together, these data suggest that increased plasma HSPB7 level after MI is likely caused by passive release instead of active synthesis from the myocardium after injury.

### Plasma HSPB7 Concentration in Acute Coronary Syndrome (ACS) Patients

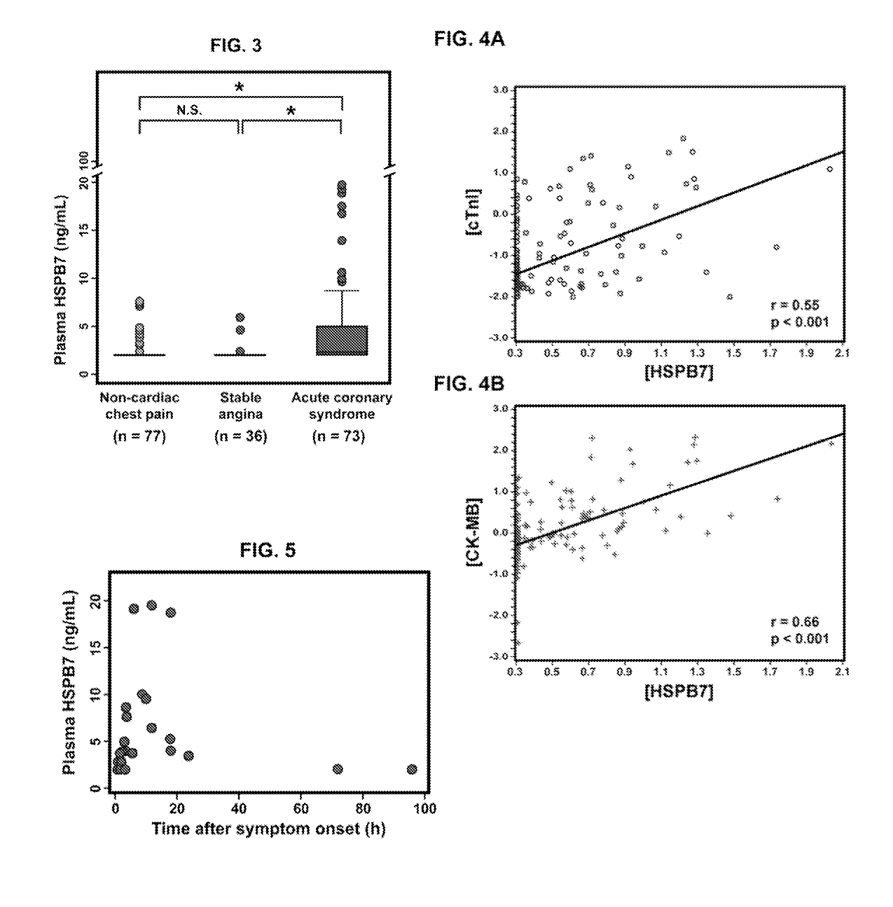

We further investigated the association of plasma HSPB7 levels and ACS in 186 patients presenting to the emergency department with chest pain: 77 patients with non-cardiac chest pain (control) group, 36 with stable angina (SA), and 73 with ACS. Table 1 lists demographic characteristics of patients. As compared with the non-cardiac chest pain group, patients in the ACS and SA groups were of similar age and sex and had similar risk factors, including diabetes mellitus, hypertension, current smoking, and CAD family history. However, the detectable rate for HSPB7 was much higher (62 versus 14 or 22%, p<0.001; Table 1) or its plasma concentration was significantly elevated in patients with ACS than in those with SA or non-cardiac chest pain (5.1 versus 2.9 ng/mL, p<0.001; FIG. 3). The ACS and non-cardiac chest pain groups differed in levels of cTnI, CK-MB, total cholesterol, low-density lipoprotein (LDL) and triglycerols, as well as leukocyte count (p<0.001 for all), whereas the SA and non-cardiac chest pain groups differed in only leukocyte count and LDL level. Furthermore, plasma HSPB7 levels were positively correlated with the concentrations of markers for myocardial necrosis (FIG. 4): cTnI (r₂=0.55, p<0.0001) or CK-MB (r₂=0.66, p<0.0001).

Table 2 shows plasma HSPB7 level among patients with acute coronary syndrome. The number of patients with ST-elevation myocardial infarction (STEMI), non-ST-elevation myocardial infarction (NSTEMI), and unstable angina (UA) are 25, 25, and 23, respectively (Table 2). The plasma HSPB7 level is detectable in more the 70 and 50 percent of patients with NSTEMI and STEMI, respectively. HSPB7 is detectable in 5 of the 23 patients with UA, as well (Table 2). The plasma level of HSPB7 correlated with cTnI, which is highest in NSTEMI group (Table 2).

TABLE 1

| | Non-cardiac chest pain | Stable angina | ACS |
|---|---|---|---|
| | (n = 77) | (n = 36) | (n = 73) |
| Age (years) | 67.2 ± 8.6 | 69.5 ± 10.3 | 69.5 ± 9.8 |
| Male, n (%) | 50 (65) | 23 (64) | 54 (74) |
| Arrival time after symptom | 11.0 ± 15.3 | 14.0 ± 19.6 | 15.1 ± 19.3 |
| onset (h) | | | |
| DM, n (%) | 25 (32) | 14 (39) | 28 (38) |
| Hypertension, n (%) | 50 (65) | 25 (69) | 42 (58) |
| Smoking, n (%) | 13 (17) | 6 (17) | 28 (38)* |
| CAD family history, n (%) | 28 (36) | 15 (42) | 26 (36) |
| Prior statin use, n (%) | 5 (7) | 7 (19) | 10 (14) |
| Laboratory tests | | | |
| HSPB7 level (ng/mL) | 2.9 ± 4.4 | 2.9 ± 4.2 | 5.1 ± 5.2^{†} |
| HSPB7 detectable rate (%) | 22.1 | 13.9 | 61.6* |
| cTnI level (ng/mL) | 0.1 ± 0.2 | 0.1 ± 0.2 | 3.6 ± 9.8* |
| CK-MB level (U/L) | 2.3 ± 2.9 | 2.1 ± 1.7 | 36.7 ± 68.5^{†} |
| Leukocyte count (cell/mm³) | 7836 ± 3144 | 6609 ± 2640^{†} | 8935 ± 3491* |
| Creatinine (mg/dL) | 1.6 ± 1.5 | 1.0 ± 0.3 | 1.2 ± 0.7 |
| Creatinine kinase level | 1.6 ± 2.2 | 1.1 ± 0.3 | 1.3 ± 0.9 |
| (mg/dL) | | | |
| Cholesterol level (mg/dL) | 165.8 ± 13.7 | 159.9 ± 26.6 | 174.8 ± 32.9* |
| LDL level (mg/dL) | 90.6 ± 18.6 | 78 ± 27.3* | 105.5 ± 33.3* |
| HDL level (mg/dL) | 42.5 ± 18.6 | 42.6 ± 9.8 | 40.2 ± 9.9 |
| Triglycerol level (mg/dL) | 162.4 ± 113.9 | 151.4 ± 89.7 | 140.4 ± 59.3* |
| Diagnosis of non-cardiac chest | | | |
| pain | n (%) | — | — |
| Myofascial pain | 25 (32.5) | | |
| Pulmonary diseases | 22 (28.6) | | |
| Cardiac diseases other than | 11 (14.3) | | |
| CAD | | | |
| GI/hepatobiliary diseases | 10 (13.0) | | |
| Anxiety | 5 (6.5) | | |
| Malignancy | 2 (2.6) | | |
| Non-specific chest pain | 2 (2.6) | | |

Data are number (%) or mean ± SD;
*p < 0.001;
^{†}p < 0.05 compare with control (non-cardiac chest pain) group.
ACS, acute coronary syndrome;
DM, diabetes mellitus;
CAD, coronary artery disease;
HSPB7, heat shock protein B7;
cTnI, cardiac troponin I;
CK-MB = creatinine kinase-MB isoform;
LDL, low density lipoprotein;
HDL, high density lipoprotein;
GI, gastrointestinal.

TABLE 2

| | STEMI | NSTEMI | Unstable angina |
|---|---|---|---|
| | (n = 25) | (n = 25) | (n = 23) |
| HSPB7 level (ng/mL) | 4.0 ± 3.6 | 9.0 ± 20.8 | 2.6 ± 1.2 |
| HSPB7 detectable rate (%) | 56.0 | 72.0 | 21.7 |
| cTnI level (ng/mL) | 2.7 ± 6.6 | 7.1 ± 14.4 | 0.05 ± 0.08 |

STEMI, ST-elevation myocardial infarction;
NSTEMI, non-ST-elevation myocardial infarction

Association of Plasma HSPB7 Concentration and Lime after Symptom Onset

Plasma HSPB7 level was detectable as early as 1 to 3 hours after the onset of symptoms in patients with ACS (FIG. 5). Most patients with a detectable HSPB7 concentration experienced a short interval between symptom onset and blood sampling, usually <24 h; however, plasma HSPB7 level was not detectable in patients presenting to the emergency department 24 h after the onset of symptoms.

### Plasma HSPB7 Concentration as a Risk Factor of ACS

Table 3 shows the results of logistic Regression Analyses of risk factors of acute coronary syndrome. Compared with traditional risk factors of CAD, plasma HSPB7 concentration was found a significantly predictor of ACS in our patient cohort (adjusted odds ratio [OR] 7.44, 95% confidence interval [CI] 1.91 to 28.93, p<0.01; Table 3). Current smoking was the only traditional risk factor predicting ACS (adjusted OR 3.54, 95% CI 1.49 to 8.42, p<0.01). Male sex, age, body mass index, hypertension, and diabetes mellitus were not predictors of ACS in our patient cohort.

HSPB7 level was detectable in some patients in the non-cardiac chest pain group. We examined whether a high HSPB7 concentration was associated with increased risk of ACS by dividing patients into 2 groups by the 75^{th} quartile of HSPB7 concentration (3.94 ng/mL). Table 4 shows odds ratios for acute coronary syndrome by levels of HSPB7. Patients with ACS were more likely than others to have high HSPB7 concentration after adjusting for other possible risk factors (adjusted OR 4.31, 95% CI 1.80-10.33, p=0.0033)

TABLE 3

| Variable | OR | 95% CI | P |
|---|---|---|---|
| Log HSPB7 (ng/mL)* | 7.44 | 1.91-28.93 | 0.0038 |
| Current smoking | 3.54 | 1.49-8.42 | 0.0042 |
| Male | 1.02 | 0.45-2.28 | 0.9667 |
| Age ≧65 y | 1.37 | 0.61-3.04 | 0.4450 |
| Body mass index ≧25 kg/m² | 1.04 | 0.50-2.17 | 0.9198 |
| Hypertension | 0.82 | 0.38-1.77 | 0.6044 |
| Diabetes mellitus | 1.19 | 0.54-2.62 | 0.6598 |
| CAD family history | 1.30 | 0.61-2.78 | 0.4980 |

*HSPB7 levels were log-transformed before analysis.
CAD, coronary artery disease;
OR, odds ratio;
95% CI, 95% confidence interval

TABLE 4

| | | | Crude OR | Adjusted OR* |
|---|---|---|---|---|
| HSPB7 | Control, n | Case, n | (95% CI) | (95% CI) |
| <3.944^{†} | 66 | 46 | 1.00 | 1.00 |
| ≧3.944 | 11 | 27 | 3.52 | 4.31 |
| | | | (1.59-7.81) | (1.80-10.33) |

p = 0.0033
OR, odds ratio;
95% CI, 95% confidence interval
*Adjusted for age, gender, smoking status, body mass index, hypertension, diabetes mellitus, and family history of coronary artery disease.
^{†}75% Q3

Because current smoking was the only traditional risk factor associated with ACS in our patient group, we then analyzed the cumulative predictive effect of current smoking and HSPB7 concentration for ACS. Table 5 shows cumulative effects of HSPB7 level and current smoking on risk of ACS. Patients with ACS were more likely than others to have both risk factors (adjusted OR 20.08, 95% CI 2.21-182.32, p=0.0077, Table 5). With either risk factor alone, the risk was lower but still significant (adjusted OR 3.87, 95% CI 1.84-8.1). Furthermore, patients with ACS were more likely than others to exhibit current smoking and high HSPB7 level (adjusted OR 18.17, 95% CI 1.96-168.77, p=0.0097). Table 6 shows combined effects of current smoking HSPB7 level on risk of ACS.

TABLE 5

| No. of | Control, | | Crude OR | Adjusted OR^{†} | |
|---|---|---|---|---|---|
| Factors* | n | Case, n | (95% CI) | (95% CI) | P |
| 0 | 54 | 26 | 1.00 | 1.00 | |
| 1 | 22 | 39 | 3.68 | 3.87 | 0.0003 |
| | | | (1.83-7.43) | (1.84-8.10) | |
| 2 | 1 | 8 | 16.62 | 20.08 | 0.0077 |
| | | | (1.97-139.94) | (2.21-182.32) | |

OR, odds ratio:
95% CI, 95% confidence interval
*The number of factors. Including high HSPB7 (>Q3, 3.944 ng/mL) and current smoking.
^{†}Adjusted for age, sex, body mass index, coronary artery disease Family history, hypertension, and diabetes mellitus.

TABLE 6

| | | | Crude OR | Adjusted OR^{†} | |
|---|---|---|---|---|---|
| Variable | Control, n | Case, n | (95% CI) | (95% CI) | P |
| Low HSPB7 + not Current | 54 | 26 | 1.00 | 1.00 | |
| Smoking | | | | | |
| Low HSPB7 + Current | 12 | 20 | 3.46 | 3.38 | 0.0044 |
| Smoking | | | (1.47-8.14) | (1.33-8.62) | |
| High HSPB7 + not Current | 10 | 19 | 3.95 | 4.14 | 0.0027 |
| Smoking | | | (1.61-9.68) | (1.59-10.73) | |
| High HSPB7 + Current | 1 | 8 | 16.62 | 18.17 | 0.0097 |
| Smoking | | | (1.97-139.94) | (1.96-168.77) | |

Low HSPB7 level: <75% (Q3; 3.944);
high HSPB7 level: ≧75% (Q3, 3.944 ng/mL).
^{†}Adjusted for age, sex, body mass index, coronary artery disease family history, hypertension, diabetes mellitus.

HSPB7 is an abundant protein selectively expressed in myocardial muscle cells. However, the biological function and clinical association of HSPB7 is not clear. In this study, we demonstrated that plasma level of HSPB7 is associated with ACS in both an animal model and human subjects. Because plasma HSPB7 concentration was detectable as early as 1 to 3 h after LAD ligation in the animal model or after MI in human patients (FIGS. 1 and 4) and because we found a positive correction between levels of plasma HSPB7 and two cardiac biomarkers of necrosis, cTnI and CK-MB (FIG. 3), HSPB7 might be rapidly released from cardiomyocytes into the circulation as a result of myocardial necrosis according to the result of Western blotting and immunohistochemical staining (FIG. 2). We found that HSPB7 level, especially high HSPB7 level, is an independent risk factor of ACS in patients presenting to the emergency department with acute chest pain. Thus, HSPB7 not only shows the potential as an early biomarker of MI but also an independent risk factor of ACS in patients with acute chest pain. This study establishes the basis for further investigating the role of HSPB7 in ACS.

The current biomarkers used to detect MI are cardiac-specific troponins (cTnI or cTnT). Measurement of cTnI or cTnT provides accurate, sensitive, and specific determination of myocardial injury but also some prognostic values and have replaced CK-MB as the preferred marker for detection of myocardial necrosis. However, troponins have some disadvantages. For example, plasma troponin concentration cannot be detected until at least 4 to 6 h after the onset of symptom. Thus, patients presenting to the emergency department with typical chest pain must undergo a second troponin measurement (usually 8 to 12 h after the onset of symptoms) if the initial measurement showed no elevation. Chest pain centers/units as a model of patient care in the emergency department was developed to decrease the cost of patient care and the adverse events. Recent multi-center studies showed that high-sensitive cTn assays can improve the early diagnosis of acute MI and risk stratification, regardless of the time of chest pain onset. Because plasma HSPB7 level is detectable as early as 1 to 3 h after the onset of symptoms (FIGS. 1 and 4), it is tempting to speculate that HSPB7 level could serve as another early myocardial necrosis biomarker which may further increase diagnostic specificity when combine with sensitive cTn assays. cTn is not elevated solely in the presence of myocardial injury. The plasma level of cTn may be elevated in sepsis, hypovolemia, atrial fibrillation, renal failure, and etc, without thrombotic event. Given that HSPB7 is only detectable in cardiac muscles, it might be utilized for the diagnosis of ACS with better specificity in some conditions mentioned above.

We examined plasma HSPB7 concentration as a risk factor for ACS instead of CAD and found that plasma HSPB7 concentration and current smoking were two independent risk factors of ACS in patients with chest pain. The cumulative effects were also significant. In the emergency department, presenting a risk factor predicting ACS is more important than presenting one predicting CAD. ACS requires emergency treatment including percutaneous coronary intervention, thrombolytic therapy, and intensive unit admission. Of note, we found HSPB7 levels in some patients with SA, and some ACS patients had elevated levels of HSPB7 but not cTnI. Therefore, HSPB7 may represent a more sensitive biomarker, at least in a sub-population of patients with low or undetectable cTnI levels, in detecting myocardial necrosis.

In conclusion, we found that plasma HSPB7 levels rise and fall rapidly after the onset of ACS in an animal model and clinical human subjects, and such levels may be an early marker of myocardial injury after MI. High HSPB7 level was significantly associated with increased risk of ACS after adjustment for traditional risk factors. Thus, plasma HSPB7 level may be an independent risk factor of ACS in patients presenting to hospital emergency departments with acute chest pain.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### REFERENCES

Anderson J L, Adams C D, Animan E M, Bridges C R, Califf R M, Casey D E, Jr., et al. ACC/AHA 2007 guidelines for the management of patients with unstable angina/non ST-elevation myocardial infarction: a report of the American College of Cardiology/American Heart Association Task Force on Practice Guidelines (Writing Committee to Revise the 2002 Guidelines for the Management of Patients With Unstable Angina/Non ST-Elevation Myocardial Infarction): developed in collaboration with the American College of Emergency Physicians, the Society for Cardiovascular Angiography and Interventions, and the Society of Thoracic Surgeons: endorsed by the American Association of Cardiovascular and Pulmonary Rehabilitation and the Society for Academic Emergency Medicine. Circulation 2007; 116: e148-304.
Cappola T P, Li M, He J, Ky B, Gilmore J, Qu L, et al. Common variants in HSPB7 and FRMD4B associated with advanced heart failure. Circ Cardiovasc Genet 2010; 3: 147-154.
Dai D F, Thajeb P, Tu C F, Chiang F T, Chen C H, Yang R B, et al. Plasma concentration of SCUBE1, a novel platelet protein, is elevated in patients with acute coronary syndrome and ischemic stroke. J Am Coll Cardiol 2008; 51: 2173-2180.
Krief S, Faivre J F, Robert P, Le Douarin B, Brument-Larignon N, Lefrere I, et al. Identification and characterization of cvHsp. A novel human small stress protein selectively expressed in cardiovascular and insulin-sensitive tissues. J Biol Chem 1999; 274: 36592-36600.
Matkovich S J, Van Booven D J, Hindes A, Kang M Y, Druley T E, Vallania F L, et al. Cardiac signaling genes exhibit unexpected sequence diversity in sporadic cardiomyopathy, revealing HSPB7 polymorphisms associated with disease. J Clin Invest 2010; 120: 280-289.
Stark K, Esslinger U B, Reinhard W, Petrov G, Winkler T, Komajda M, et al. Genetic association study identifies HSPB7 as a risk gene for idiopathic dilated cardiomyopathy. PLoS Genet 2010; 6: e1001167.
Tarnavski O, McMullen J R, Schinke M, Nie Q, Kong S & Izumo S Mouse cardiac surgery: comprehensive techniques for the generation of mouse models of human diseases and their application for genomic studies. Physiol Genomics 2004; 16: 349-360.

What is claimed is:

1. A method for determining an increased risk of developing a cardiovascular event excluding stable angina in a subject, comprising:
obtaining a plasma sample from the subject;
measuring a plasma level of heat shock protein B7 (HSPB7) in the sample;
comparing the plasma level of HSPB7 in the sample with a control; and
determining that the subject has an increased risk for developing the cardiovascular event excluding stable angina if the plasma level of HSPB7 in the sample is higher than the control.

2. The method of claim 1, wherein the measuring step is performed within 3 hours from receiving a complaint of chest pain from the subject.

3. The method of claim 2, comprising determining that the subject has an increased risk for developing acute coronary syndrome (ACS) if the plasma level of HSPB7 in the sample is ≧5.1 ng/mL.

4. The method of claim 1, wherein the measuring step is performed within 20 hours from receiving a complaint of chest pain from the subject, and wherein the cardiovascular event is myocardial infarction.

5. The method of claim 1, wherein the measuring step is performed within 12 hours from receiving a complaint of chest pain from the subject.

6. The method of claim 1, further comprising:
measuring a plasma level of cardiac troponin I (cTnI) or creatinine kinase-MB isoform (CK-MB), or both;
comparing the plasma level of cTn1 or CK-MB with corresponding data from a control; and
identifying the subject as having ACS if the plasma levels of cTn1, CK-M or both are higher than the control.

7. The method of claim 1, wherein the plasma level of HSPB7 in the sample is ≧2 ng/mL.

8. The method of claim 1, the cardiovascular event is selected from the group consisting of atherosclerotic vascular disease, myocardial infarction, acute cardiac syndrome, stroke, a transient ischemic attack, and critical limb ischemia.

9. The method of claim 1, wherein the measuring step is performed within 6 hours from receiving a complaint of chest pain from the subject.

10. The method of claim 1, wherein the measuring step is performed with an enzyme-linked immunosorbent assay (ELISA) using anti-human HSPB7 monoclonal antibody (mAb).

11. The method of claim 10, wherein the plasma level of HSPB7 in the sample is ≧2 ng/mL.

12. The method of claim 1, wherein the anti-hHSP7 mAb recognizes an epitope located in amino acid residues 2-170 of a human HSPB7.

13. A method for diagnosing a human subject's myocardial infarction (MI) and/or acute coronary syndrome (ACS) state or identifying a human subject's risk of MI and/or ACS, the method comprising:
a) measuring the level of heat shock protein B7 (HSPB7) in a plasma sample from said human subject;
b) comparing the level of heat shock protein B7 (HSPB7) in the plasma sample from said subject to data obtained from one or more than one reference plasma sample from a control human subject to identify an increase or decrease in the level of HSPB7 in the plasma sample from said subject; and
c) using said increase or decrease in the level of HSPB7 in the plasma sample from said human subject to diagnose the human subject's MI and/or ACS state or change in MI and/or ACS health state, or to identify the risk of MI and/or ACS in said subject, wherein the human subject is diagnosed with MI and/or ACS or an increased risk of MI and/or ACS based on having an elevated level of HSPB7.

14. The method of claim 13, wherein the measuring step is performed within 3 hours from receiving a complaint of chest pain from the subject.

15. The method of claim 14, wherein the analyzing step further comprises measuring the level of cardiac troponin 1 (cTn1) or creatinine kinase-MB isoform (CK-MB), or both, and wherein the human subject is diagnosed with MI and/or ACS based on having an elevated level of cTn1 or CK-MB, or both.

16. The method of claim 17, wherein the measuring step is performed under 6-12 hours from receiving a complaint of chest pain from the subject.

17. A method for diagnosing and/or predicting ACS in a human subject, comprising:
measuring a plasma level of HSPB7 in the human subject;
comparing the plasma level of HSPB7 in the human subject to the plasma level of HSPB7 of a control human subject; and
diagnosing or predicting relative risk for the development of ACS in said human subject based on having an elevated level of HSPB7.

18. The method of claim 17, wherein the measuring step is performed under 24 hours from receiving a complaint of chest pain from the subject.

19. The method of claim 17, wherein the measuring step is performed within 1-3 hours from receiving a complaint of chest pain from the subject.

20. The method of claim 1, wherein the subject is free of any one of the following conditions or diseases: idiopathic cardiomyopathic conditions, significant valvular heart disease, any malignancy, hematologic or rheumatologic disease, and chronic kidney disease.


SEQUENCE LISTING (3 sequences)

SEQ ID NO: 1 - protein, 170 aa, Homo sapiens
MSHRTSSTFRAERSFHSSSSSSSSSTSSSASRALPAQDPPMEKALSMFSDDFGSFMRPHSEPLAFPARPGGAGNIKTLGDAYEFAVDVRDFSPEDIIVTTSNNHIEVRAEKLAADGTVMNTFAHKCQLPEDVDPTSVTSALREDGSLTIRARRHPHTEHVQQTFRTEIKI

SEQ ID NO: 2 - protein, 169 aa, Mus musculus
MSHRTSSAFRAERSFRSSSSSSSSSSSSASRALPAQDPPMEKALSMFSDDFGSFMLPHSEPLAFPARPGGQGNIKTLGDAYEFTVDMRDFSPEDIIVTTFNNHIEVRAEKLAADGTVMNTFAHKCQLPEDVDPTSVTSALREDGSLTIRARRHPHTEHVQQTFRTEIKI

SEQ ID NO: 3 - protein, 161 aa, Danio rerio
MSASNSSAYRSERSYHQTSSSSSSSSTSANPYMEKSRGLFADDFGSFMCPKDALGFPNRTGTVGNIKTLGDTYQFTVDVQDFSPEDVIVTTSNNQIEVHAEKLASDGTVMNTFTHKCRLPEDVDPTSVKSSLGADGTLTIKAQRNTAKLEHAQTFRTEIKI